(19) United States
(12) Patent Application Publication
(10) Pub. No.: US 2025/0205639 A1
(43) Pub. Date: Jun. 26, 2025

(54) CARBON DIOXIDE SEPARATOR, AND METHOD FOR SEPARATING OR RECOVERING CARBON DIOXIDE

(71) Applicant: Research Institute of Innovative Technology for the Earth, Kyoto (JP)

(72) Inventors: Firoz Alam Chowdhury, Kyoto (JP); Thi Quyen Vu, Kyoto (JP); Katsunori Yogo, Kyoto (JP)

(21) Appl. No.: 18/849,662

(22) PCT Filed: Feb. 7, 2023
(86) PCT No.: PCT/JP2023/003976

§ 371 Date: Sep. 23, 2024

(30) Foreign Application Priority Data
Mar. 25, 2022 (JP) 2022-050153

(51) Int. Cl. B01D 53/62; B01D 53/81; B01D 53/96; B01J 20/26; B01J 20/28; B01J 20/34; C01B 32/50; C07D 295/13

(52) U.S. Cl. CPC B01D 53/62; B01D 53/81; B01D 53/96; B01J 20/262; B01J 20/2803; B01J 20/28059; B01J 20/28061; B01J 20/28064; B01J 20/28071; B01J 20/28073; B01J 20/28076; B01J 20/3425; B01J 20/3466; B01J 20/3483; B01J 20/3491; C01B 32/50; C07D 295/13; B01D 2252/20415; B01D 2257/504; B01D 2259/4009

(57) ABSTRACT

A polyamine including a ring-containing polyamine having a piperazine ring. At least one of two nitrogen atoms of the piperazine ring is bonded to a chain substituent represented by -(A1-NR1)ₘ-X, where m represents an integer of 2 to 50, A1 represents an alkylene group having 2 to 6 carbon atoms, a plurality of A1 may be identical or different from each other, R1 is a hydrogen atom, an alkyl group having 1 to 6 carbon atoms, or an alkylamino group having 1 to 6 carbon atoms, a plurality of R1 may be identical or different from each other, and at least one R is a hydrogen atom or an alkylamino group having 1 to 6 carbon atoms, and X is a hydrogen atom, an alkyl group having 1 to 6 carbon atoms, or an alkylamino group having 1 to 6 carbon atoms.

### TECHNICAL FIELD

The present invention relates to a carbon dioxide separating material and a method for separating or recovering carbon dioxide.

### BACKGROUND ART

Patent Literature 1 proposes a carbon dioxide separating material including a polyamine-containing material in which a polyamine having at least two isopropyl groups on one or more of nitrogen atoms is carried on a support, and a method for separating or recovering carbon dioxide using the carbon dioxide separating material.

Patent Literature 2 proposes a method for preparing alkylalkanolamines, the method including a step of reacting a carbonyl compound with a hydroxyl alkyl amine in the presence of hydrogen and catalyst.

Patent Literature 3 proposes a core-shell type amine-based carbon dioxide adsorbent having, as a core, a porous support with an amine compound immobilized thereon and, as a shell, an amine layer resistant to inactivation by sulfur dioxide, and including a chelating agent that inhibits oxidative decomposition of amine and has resistance to oxygen and sulfur dioxide.

Patent Literature 4 proposes a regenerable solid sorbent including a modified polyamine and a solid support, and configured to adsorb carbon dioxide from a gas mixture including air. The modified polyamine is the reaction product of an amine and an epoxide.

### CITATION LIST

### Patent Literature

Patent Literature 1: International Publication WO2014/208712
Patent Literature 2: Japanese Laid-Open Patent Publication No. 2012-530771 (Japanese Patent No. 5678050)
Patent Literature 3: U.S. Pat. No. 10,654,025
Patent Literature 4: U.S. Patent Publication No. 2019/0168185

### SUMMARY OF INVENTION

### Technical Problem

There have been developed a plurality of materials for adsorbing carbon dioxide as mentioned above, for which, however, further improvements in performance are still desired. For example, it is desired to improve the resistance to oxidative deterioration of a carbon dioxide separating material including a support and a polyamine carried thereon. This is because the carbon dioxide separating material is assumed to be used in an oxygen-containing atmosphere (esp. air) at a temperature of, for example, about 60° C.

### Solution to Problem

One aspect of the present invention relates to a polyamine, including a ring-containing polyamine having a piperazine ring, wherein
at least one of two nitrogen atoms of the piperazine ring is bonded to a chain substituent represented by

-(A1-NR1)ₘ-X,
where m represents an integer of 2 to 50,
A1 represents an alkylene group having 2 to 6 carbon atoms,
a plurality of A1 may be identical or different from each other,
R1 is a hydrogen atom, an alkyl group having 1 to 6 carbon atoms, or an alkylamino group having 1 to 6 carbon atoms,
a plurality of R1 may be identical or different from each other, and at least one R is a hydrogen atom or an alkylamino group having 1 to 6 carbon atoms, and
X is a hydrogen atom, an alkyl group having 1 to 6 carbon atoms, or an alkylamino group having 1 to 6 carbon atoms.

Another aspect of the present invention relates to a carbon dioxide separating material, including the above-described polyamine, and a support carrying the polyamine.

Still another aspect of the present invention relates to a method for separating or recovering carbon dioxide, including:
a first step of bringing a target gas into contact with the above-described carbon dioxide separating material, to allow carbon dioxide to be absorbed into the carbon dioxide separating material; and
a second step of releasing carbon dioxide from the carbon dioxide separating material into which the carbon dioxide has been absorbed in the first step, wherein
the second step includes one or more of:
(A) a process of placing the carbon dioxide separating material under reduced pressure conditions, to release carbon dioxide (pressure swing method),
(B) a process of bringing at least one of water vapor and an inert gas into contact with the carbon dioxide separating material, to release carbon dioxide, and
(C) a process of heating the carbon dioxide separating material, to release carbon dioxide (temperature swing method).

### Advantageous Effects of Invention

A polyamine according to the present disclosure has high resistance to oxidative deterioration and is excellent in carbon dioxide adsorption-desorption performance. By using a carbon dioxide separating material including the polyamine according to the present disclosure, it is possible to separate or recover carbon dioxide with high efficiency over a long term.

While the novel features of the invention are set forth particularly in the appended claims, the invention, both as to organization and content, will be better understood and appreciated, along with other objects and features thereof, from the following detailed description taken in conjunction with the drawings.

### BRIEF DESCRIPTION OF DRAWINGS

FIG. 1 A set of graphs showing the relationship between the amine efficiency and the partial pressure of carbon dioxide.

FIG. 2 A set of graphs showing the relationship between the partial pressure and the adsorption amount of carbon dioxide up to an equilibrium pressure of 0.1 kPa.

FIG. 3 A set of graphs showing the relationship between the partial pressure and the adsorption amount of carbon dioxide up to an equilibrium pressure of 100 kPa.

FIG. 4 A graph showing the carbon dioxide adsorption ability before and after oxidative deterioration of carbon dioxide separating materials.

FIG. 5 A set of graphs showing IR spectra before and after oxidative deterioration of carbon dioxide separating materials.

FIG. 6 A graph showing the kinetic behavior of adsorption and release of carbon dioxide by carbon dioxide separating materials.

FIG. 7 A graph showing the temperature dependence of carbon dioxide release process using carbon dioxide separating materials containing a polyamine having at least one isopropyl group and a carbon dioxide separating material containing a polyamine having no isopropyl group.

FIG. 8 A set of graphs showing the relationship between the partial pressure and the adsorption amount of carbon dioxide up to an equilibrium pressure of 0.2 kPa or 100 kPa, when the carbon dioxide adsorption temperature is changed.

### DESCRIPTION OF EMBODIMENTS

A carbon dioxide separating material according to an embodiment of the present invention will be described below by way of examples, but the carbon dioxide separating material is not limited to the examples below. In the following description, specific numerical values and materials are exemplified in some cases, but other numerical values and other materials may be adopted as long as the effects of the present disclosure can be obtained. In the present specification, the phrase “a numerical value A to a numerical value B” includes the numerical value A and the numerical value B, and can be rephrased as “a numerical value A or more and a numerical value B or less. In the following description, when the lower and upper limits of numerical values related to specific physical properties, conditions, etc. are mentioned as examples, any one of the mentioned lower limits and any one of the mentioned upper limits can be combined in any combination as long as the lower limit is not equal to or more than the upper limit. When a plurality of materials are mentioned as examples, one kind of them may be selected and used singly, or two or more kinds of them may be used in combination.

Hereinafter, the term “polyamine” means either a polyamine consisting of a single amine compound or a mixture of a plurality of amine compounds, depending on the context. In the present invention, the term “contains” or “includes” is an expression that encompasses “contains (or includes),” “substantially consists of,” and “consists of.”

The polyamine according to the present disclosure (hereinafter sometimes referred to as a “polyamine (P)”) includes, at least, a ring-containing polyamine having a piperazine ring. A chain substituent is bonded to at least one of the two nitrogen atoms of the piperazine ring. When a chain substituent is bonded to only one of the two nitrogen atoms of the piperazine ring, a hydrogen atom is bonded to the other nitrogen atom.

The chain substituent is represented by -(A1-NR1)ₘ-X. The symbol m represents an integer of 2 to 50. A1 represents an alkylene group having 2 to 6 carbon atoms. A plurality of A1 may be identical or different from each other. R1 is a hydrogen atom, an alkyl group having 1 to 6 carbon atoms, or an alkylamino group having 1 to 6 carbon atoms. A plurality of R1 may be identical or different from each other. However, at least one R1 is a hydrogen atom or an alkylamino group having 1 to 6 carbon atoms. X is a hydrogen atom, an alkyl group having 1 to 6 carbon atoms, or an alkylamino group having 1 to 6 carbon atoms. Hereinafter, the ring-containing polyamine having the above characteristics is sometimes referred to as a “ring-containing polyamine (PP).”

In the chain substituent -(A1-NR1)ₘ-X, X may be, for example, a hydrogen atom or an isopropyl group. The introduction of an isopropyl group weakens the chemical bond between N and CO₂, making it possible to release carbon dioxide from the carbon dioxide separating material with less energy (e.g., at low temperature). This allows for efficient recovery of carbon dioxide.

In the chain substituent -(A1-NR1)ₘ-X, m may be an integer of 2 to 6, and may be an integer of 2 to 4.

When a plurality of A1 is identical, A1 may be an ethylene group (—CH₂CH₂—) having 2 carbon atoms or a propylene group (—CH₂CH₂CH₂—) having 3 carbon atoms.

When a plurality of R1 is identical, R1 may be a hydrogen atom. That is, the —NR1- group may be an —NH— group. For example, the —NH— group may generate a carbamate through the following reaction.

2=NH+CO₂→═NH₂⁺+═NCOO⁻

The polyamine (P) may include, as the ring-containing polyamine (PP), for example, at least one selected from the group consisting of ring-containing polyamines represented by the following formulas (1) to (5).

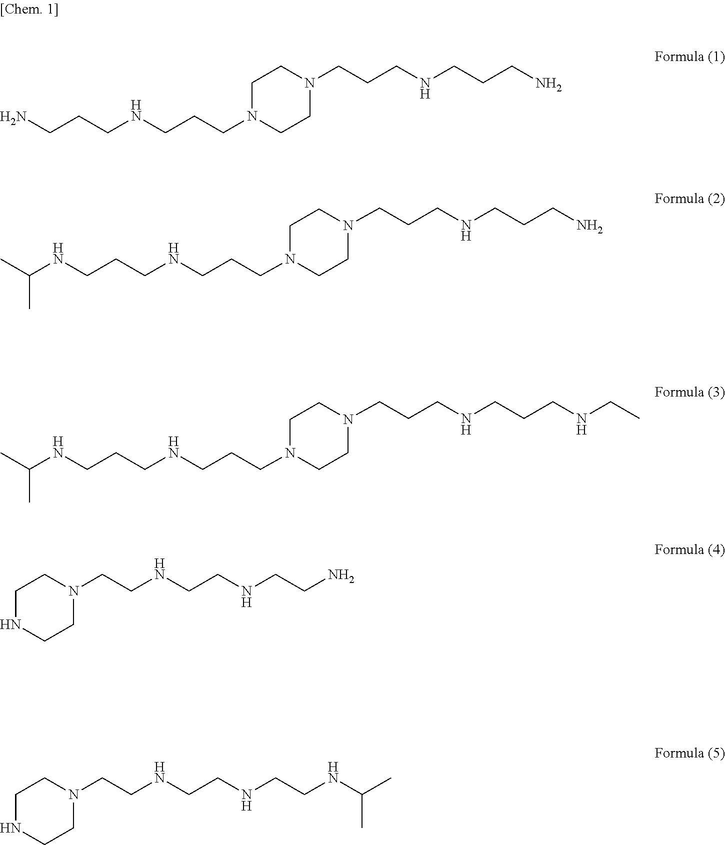 [SMILES: NCCCNCCCN1CCN(CCCNCCCN)CC1 ; CC(C)NCCCNCCCN1CCN(CCCNCCCN)CC1 ; CCNCCCNCCCN1CCN(CCCNCCCNC(C)C)CC1 ; NCCNCCNCCN1CCNCC1 ; CC(C)NCCNCCNCCN1CCNCC1]

Hereinafter, five types of the ring-containing polyamines (PP) represented by any one of the above formulas (1) to (5) are also collectively referred to as ring-containing polyamines (PP5).

In the chain substituent -(A1-NR1)ₘ-X, when at least a part of X is an isopropyl group, for more efficient recovery of carbon dioxide, the ratio IP group/PP of the number of moles of the isopropyl group (IP group) included in the ring-containing polyamine (PP) to the number of moles of the ring-containing polyamine (PP) may be, for example, 0.25 to 0.75, and may be 0.37 to 0.64.

The polyamine (P) can include a polyamine other than the ring-containing polyamine (PP). However, for enhancing the resistance of the polyamine (P) to oxidative deterioration, the content of the ring-containing polyamine (PP) in the polyamine (P) is desirably more than 6 mass %, more preferably 15 mass % or more, further more preferably 21 mass % or more.

For further enhancing the resistance to oxidative deterioration of the polyamine (P), the content of the ring-containing polyamines (PP5) in polyamine (P) is preferably more than 6 mass %, more preferably 18 mass % or more, further more preferably 25 mass % or more. In addition, in the ring-containing polyamines (PP5), the polyamine represented by the formula (1) may be the major component, the polyamine represented by the formula (2) may be the major component, the polyamine represented by the formula (3) may be the major component, the polyamine represented by the formula (4) may be the major component, or the polyamine represented by the formula (5) may be the major component. Here, the major component in the ring-containing polyamines (PP5) means that the content of the major one among the polyamines (1) to (5) relative to the total amount of the ring-containing polyamines (PP5) contained in polyamine (P) is 50 mass % or more, or even 70 mass % or more. It can be also read that the content of the polyamine represented by the formula (1) in the polyamine (P) may be 50 mass % or more, or even 70 mass %, the content of the polyamine represented by the formula (2) in the polyamine (P) may be 50 mass % or more, or even 70 mass %, the content of the polyamine represented by the formula (3) in the polyamine (P) may be 50 mass % or more, or even 70 mass %, the content of the polyamine represented by the formula (4) in the polyamine (P) may be 50 mass % or more, or even 70 mass %, or the content of the polyamine represented by the formula (5) in the polyamine (P) may be 50 mass % or more, or even 70 mass %.

The ring-containing polyamine (PP) can be synthesized using, as a raw material, for example, a piperazine ring-containing compound, such as piperazine, N-monoalkyleneamino piperazine having a terminal amino group, and N,N′-dialkyleneamino piperazine having terminal amino groups. For example, the piperazine ring-containing compound may be allowed to react with a reactant, such as an imine compound and an aziridine compound, under predetermined conditions. The reactant may be at least one selected from the group consisting of ethyleneimine, propyleneimine, 2-ethylaziridine, 2-propylaziridine, and 2-butylaziridine. Alternatively, for example, the piperazine ring-containing compound, such as N-(2-aminoethyl)piperazine, N-(3-aminopropyl)piperazine, N,N′-bis(2-aminoethyl)piperazine, and N,N′-bis(3-aminopropyl)piperazine, may be subjected to an addition reaction with acrylonitrile and, thereafter, reduction of the nitrile group (—CN) with hydrogen.

The polyamine (P) may include, in addition to the ring-containing polyamine (PP), a chain polyamine. The chain polyamine may have a structure represented by Y-(A2-NR2)ₘ-Y. Here, m represents an integer of 2 to 50. A2 represents an alkylene group having 2 to 6 carbon atoms. A plurality of A2 may be identical or different from each other. R2 represents a hydrogen atom, an alkyl group having 1 to 6 carbon atoms, or an alkylamino group having 1 to 6 carbon atoms. A plurality of R2 may be identical or different from each other. However, at least one R2 is a hydrogen atom or an alkylamino group having 1 to 6 carbon atoms. Y represents a hydrogen atom, an alkyl group having 1 to 6 carbon atoms, or an alkylamino group having 1 to 6 carbon atoms. The two Y's may be identical or different from each other.

Hereinafter, a chain polyamine having the above characteristics is sometimes referred to as a “chain polyamine (CP).”

In the chain polyamine (CP), Y may be, for example, a hydrogen atom or an isopropyl group. The introduction of an isopropyl group weakens the chemical bond between N and CO₂, making it possible to release carbon dioxide from the carbon dioxide separating material with less energy (e.g., at low temperature). This allows for efficient recovery of carbon dioxide.

In the chain polyamine (CP), m may be an integer of 2 to 6, and may be an integer of 2 to 4.

When a plurality of A2 is identical, A2 may be an ethylene group (—CH₂CH₂—) having 2 carbon atoms or a propylene group (—CH₂CH₂CH₂—) having 3 carbon atoms.

When a plurality of R2 is identical, R2 may be a hydrogen atom. That is, the —NR₂— group may be an —NH— group.

The ratio IP group/PP of the number of moles of the isopropyl group (IP group) included in the chain polyamine (CP) to the number of moles of the chain polyamine (CP) may be, for example, 0.10 to 0.50, and may be 0.25 to 0.75.

The difference between the formula weight of the ring-containing polyamine (PP) and the formula weight of the chain polyamine (CP) is preferably equivalent to the formula weight of an ethylene group (—CH₂CH₂—). Such a polyamine (P) is relatively easily manufactured, purified, etc.

The backbone amine or raw material amine of the chain polyamine (CP) may be, for example, at least one selected from the group consisting of homopolymers of ethyleneimine, propyleneimine, 2-ethylaziridine, 2-propylaziridine, and 2-butylaziridine, and copolymers of at least two of these. Here, the homopolymers and copolymers include oligomers in which the number of polymerized molecules is 10 or less (e.g., 7 or less).

Specific examples of the backbone amine include tetraethylenepentamine, spermine, pentaethylenehexamine, hexaethyleneheptamine, triethylenetetramine, N,N,N′,N′-tetrakis(3-aminopropyl)-1,4-butanediamine, 1,11-diamino-3,6,9-triazaundecane, N,N′-bis(3-aminopropyl)-1,4-butanediamine, N,N,N′,N′-tetrakis(3-aminopropyl)-1,4-butanediamine, 1,14-diamino-3,6,9,12-tetraazatetradecane, 1,17-diamino-3,6,9,12,15-pentaazaheptadecane, and 1,8-diamino-3,6-diazaoctane.

The backbone amine may be, for example, at least one selected from the group consisting of tetraethylenepentamine, spermine, N,N,N′,N′-tetrakis(3-aminopropyl)-1,4-butanediamine, pentaethylenehexamine, hexaethyleneheptamine, and triethylenetetramine.

The boiling point of the polyamine (P) is desirably 320° C. or higher at 760 mmHg. In this case, the carbon dioxide separating material can be used stably even at high temperatures (e.g., at about 60° C.). Provided that the boiling point is 320° C. or higher at 760 mmHg, the polyamine (P) can be maintained in the state of being carried on the support, even though the boiling point is lowered under reduced pressure (e.g., about 0.2 Pa). Therefore, when the polyamine (P) is used, the operating temperature can be set higher than room temperature, and the release of carbon dioxide can be efficiently performed. The upper limit of the boiling point of the polyamine (P) is not particularly limited, but may be, for example, about 500° C. at 760 mmHg.

In the ring-containing polyamine (PP) and the chain polyamine (CP), when X or Y is an isopropyl group, the isopropyl group may be introduced by, for example, allowing a starting material of the isopropyl group to react with the amino group of a raw material polyamine, in the following manner. As the starting material of the isopropyl group, for example, acetone may be used.

The raw material polyamine may be, for example, a predetermined polyamine having a —NH₂ group and a —NH— group, which is commercially available or obtained by a known method.

As a method for introducing an isopropyl group to a terminal of the molecule, for example, a —NH₂ group may be allowed to react with a starting material, such as acetone. At this time, the molar ratio between the raw material polyamine and the acetone may be controlled, to control the ratio IP group/PP ratio in the ring-containing polyamine (PP), or the ratio IP group/PP in the chain polyamine (CP).

Specifically, a platinum oxide catalyst and anhydrous ethanol are placed in a reaction vessel, such as a flask, and after the atmosphere in the reaction vessel is replaced with hydrogen, hydrogen is added until a pressure of 100 kPa to 150 kPa is reached, followed by stirring for a predetermined time, to reduce the platinum oxide catalyst. Next, a raw material polyamine, acetone, and anhydrous ethanol are placed in the reaction vessel containing the reduced catalyst, and after the atmosphere in the reaction vessel is replaced with hydrogen, hydrogen is added until a pressure of about 200 kPa to 350 kPa is reached, followed by stirring while hydrogen is supplied, until no drop in pressure is observed. At this time, the N═C bond formed by a dehydration reaction between acetone and —NH₂ is hydrogenated. After the solution is filtered to remove the catalyst therefrom, the ethanol is removed under reduced pressure, and the resulting colorless liquid is further dried under vacuum, and then, a polyamine (PP, CP) having an isopropyl group can be obtained. Acetone reacts more preferentially with primary amino groups than with secondary amino groups. Therefore, the isopropyl group is preferentially bonded to a terminal of the molecule.

The carbon dioxide separating material may include a polyamine-containing material including a polyamine (P) and a support carrying the polyamine (P).

### <Support>

The support may be any material that can carry a polyamine (P) and withstand the conditions for separating and recovering carbon dioxide. For example, ceramics, porous materials, carbon materials, resin materials, and the like can be used. Specific examples thereof include silica, polymethyl methacrylate, alumina, silica alumina, clay minerals, cordierite, magnesia, zirconia, zeolite, zeolite-related compounds, natural minerals, waste solids, activated carbon, cellulose, and carbon molecular sieves. The support may be used singly or in combination of two or more kinds.

As the support, a commercially available product may be used as it is, or a support synthesized by a known method may be used. Examples of the commercially available product include mesostructured silica MSU-F manufactured by Sigma-Aldrich Co. LLC, SIPERNAT (registered trademark) 50S manufactured by Evonik Industries, and CARiACT (registered trademark) Q10, Q30, Q50 manufactured by Fuji Silysia Chemical Ltd.

The support is preferably a porous material having a large specific surface area and a large pore volume, for allowing a large amount of the polyamine (P) to be carried thereon. The specific surface area (BET) is desirably 50 m²/g or more and 2000 m²/g or less, more desirably 100 m²/g or more and 1000 m²/g or less. The pore volume is desirably 0.1 cm³/g or more and 2.3 cm³/g or less, more desirably 0.7 cm³/g or more and 2.3 cm³/g or less.

The specific surface area and the pore volume can be measured by, for example, a constant volume method using a specific surface area-pore size distribution analyzer (ASAP2420 manufactured by Shimadzu Corporation). As a specific method for measuring gas adsorption using a specific surface area-pore size distribution analyzer, for example, a pretreatment of a sample is performed by evacuation under heating, and about 0.1 g of the sample for measurement is weighed into a sample tube. This is followed by heating to 40° C., and evacuating for 6 hours. After cooling to room temperature, the sample mass is measured. In the measurement, the liquid nitrogen temperature is preset, and the pressure range is designated. The specific surface area, the pore volume, and the pore diameter can be calculated by analyzing the resulting nitrogen adsorption isotherm.

### <Polyamine-Containing Material>

The polyamine-containing material is a carbon dioxide separating material in which a polyamine (P) is carried on a support.

The method for producing a polyamine-containing material includes a step of preparing a polyamine (P), and a step of obtaining a polyamine-containing material. In the step of obtaining a polyamine-containing material, the polyamine (P) is brought into contact with a support, to allow the polyamine (P) to be carried on the support.

The polyamine-containing material can be produced by, for example, mixing a support with a polyamine (P) solution, and stirring the mixture, for example, at room temperature, followed by distilling off the solvent (e.g., water, alcohol). As a method for distilling off the solvent, for example, a method of reducing the pressure under heating with an evaporator or the like can be used.

By carrying the polyamine (P) on the support, applications are possible to a pressure swing method and a temperature swing method, to which a carbon dioxide separating material in the form of aqueous solution cannot be applied. The pressure swing method includes a step of placing the carbon dioxide separating material under reduced pressure conditions, to release carbon dioxide. The temperature swing method includes a step of heating the carbon dioxide separating material, to release carbon dioxide.

### <Carbon Dioxide Separating Material>

The carbon dioxide separating material includes, for example, a polyamine-containing material, and a binder for granulating the polyamine-containing material. That is, the carbon dioxide separating material may include a polyamine-containing material as a granulated product using a binder. By granulating the polyamine-containing material using a binder, the vibration resistance and the abrasion resistance can be imparted thereto, and the stability in water can be improved.

The binder may be at least one selected from the group consisting of silica, alumina, silica alumina, clay minerals, fluorocarbon resins, cellulose derivatives, and epoxy resins. Examples of the fluorocarbon resins include polytetrafluoroethylene. Examples of the cellulose derivatives include hydroxypropylmethyl cellulose, methyl cellulose, hydroxypropyl cellulose, carboxymethyl cellulose, hydroxyethyl cellulose, and hydroxyethylated starch. Examples of the epoxy resins include diglycerol polyglycidyl ether, and sorbitol polyglycidyl ether, each of which may be used as a mixture with a curing agent for epoxy resins (modified polyamide resin, etc.). Other polymers (polyvinyl alcohol, polyethylene oxide, sodium polyacrylate, polyacrylamide, etc.) may also be used. These compounds are commercially available or can be easily produced by known methods. The binder may be used singly or in combination of two or more kinds.

Examples of the commercially available binders include SNOWTEX-30 and AS-200 manufactured by Nissan Chemical Corporation, POLYFLON PTFED-210C manufactured by Daikin Industries, Ltd., NEOVISCO MC RM4000 manufactured by Sansho Co., Ltd., AQ Nylon P-70 manufactured by Toray Industries, Inc., and DENACOL EX-421 manufactured by Nagase ChemteX Corporation.

The amount of the binder contained in the carbon dioxide separating material is not particularly limited as long as granulation is possible with that amount, but is preferably small in order to avoid the decrease of the polyamine content.

When the granulation is performed using a binder, the average particle diameter of the granulated product is preferably 0.1 mm to 2.0 mm, in view of reducing the pressure loss when gas is supplied to the adsorbent-packed layer.

The content of the polyamine (P) in the carbon dioxide separating material is not particularly limited, but in view of efficiently separating and recovering carbon dioxide, for example, is preferably 15 mass % or more, particularly preferably 20 mass % or more. The content of the polyamine (P) may be, for example, 70 mass % or less.

### <Method for Separating or Recovering Carbon Dioxide>

The target to be treated by the carbon dioxide separation (recovery) method is a carbon dioxide-containing gas. The carbon dioxide-containing gas may be, for example, an exhaust gas discharged from: thermal power plants using coal, heavy oil, natural gas, and other fuels, blast furnaces of ironworks where iron oxide is reduced with coke; converters of ironworks where carbon in pig iron is combusted to produce steel; factory boilers; cement plant kilns; and transportation equipment, such as automobiles, ships, and aircraft, using gasoline, heavy oil, light oil, or other fuels. The carbon dioxide-containing gas may be a gas containing carbon dioxide, etc. produced by human breathing, energy conversion by equipment, and other actions, in an enclosed space, such as a submersible research vessel, a space station, and the indoor space of building, office, etc. Also, it may be carbon dioxide in the atmosphere.

The method for separating (recovering) carbon dioxide includes a first step of bringing a target gas into contact with the carbon dioxide separating material, to allow carbon dioxide to be absorbed into the carbon dioxide separating material; and a second step of releasing carbon dioxide from the carbon dioxide separating material into which the carbon dioxide has been absorbed in the first step.

The carbon dioxide content and the temperature of the target gas in the first step are not particularly limited as long as the carbon dioxide separating material can withstand those conditions. For example, the carbon dioxide partial pressure may be 100 kPa or less, and the temperature may be −5° C. to 100° C. Specifically, the conditions may be those assumed to be used in thermal power plants etc. (carbon dioxide partial pressure: 3 to 100 kPa, temperature: 40 to 80° C.), or those assumed to be used in space stations etc. (carbon dioxide partial pressure: 1 kPa or less, temperature: 1 to 40° C.). The target gas may be at atmospheric pressure or may be pressurized.

The target gas in the first step may contain water vapor. The carbon dioxide separating material exhibits excellent carbon dioxide adsorption properties even when the target gas contains water vapor, and therefore, the dehumidification operation can be omitted.

The method of releasing carbon dioxide in the second step may be a method including a process (A) of placing the carbon dioxide separating material under reduced pressure conditions, to release carbon dioxide (pressure swing method), a process (B) of bringing at least one of water vapor and an inert gas (preferably a carbon dioxide-free gas (or a gas with low carbon dioxide content)) into contact with the carbon dioxide separating material, to release carbon dioxide, a process (C) of heating the carbon dioxide separating material, to release carbon dioxide (temperature swing method), and the like.

In the method including the process (A), the pressure is preferably reduced to about 0.2 Pa, in view of the amount of the carbon dioxide released and the stability of the carbon dioxide separating material. During the pressure reduction, the carbon dioxide separating material or a container containing the carbon dioxide separating material may be heated. When heating, desirably, the temperature is up to about 60° C., and in this case, the pressure is preferably reduced to about 0.5 Pa. The method including the process (A) is suitable when the target gas has a temperature of 10 to 60° C. and a carbon dioxide partial pressure of 100 kPa or less.

In the method including the process (B), for example, by bringing an inert gas, water vapor, a carbon dioxide-free gas, or the like into contact with the carbon dioxide separating material, the partial pressure of carbon dioxide can be reduced, and carbon dioxide can be released. The gas to be brought into contact with the carbon dioxide separating material may be any gas as long as the carbon dioxide separating material can be stable in that gas, which is preferably an inert gas like as argon, nitrogen, water vapor, and the like, more preferably a water vapor with reduced pressure.

In the method including the process (C), by raising the temperature to be higher than the temperature at the time of carbon dioxide absorption, carbon dioxide can be released. In this case, the temperature at the time of carbon dioxide absorption may be, for example, 10 to 40° C., and the temperature at the time of carbon dioxide release may be, for example, about 60° C.

### EXAMPLES

Next, the present invention will be specifically described with reference to Examples. The present invention, however, is not limited to the following Examples. The polyamine carried amount (mass %) refers to a mass of the polyamine expressed in percentage relative to the mass of the carbon dioxide separating material excluding carbon dioxide (here, the ratio of the polyamine to the total of the polyamine and the support).

For measuring the physicochemical properties of a synthesized polyamine, the following apparatus was used.

Liquid chromatograph mass spectrometer (LC-MS): Alliance LC/MS system manufactured by Nihon Waters K.K.

In the following, the expressions “xIP” etc. in “xIP-TEPA” etc. indicate that the number of moles of acetone allowed to react with 1 mole of the backbone amine (TEPA in this example) is x mol. For example, the expressions “2.0IP” etc. in “2.0IP-TEPA” etc. indicate that the number of moles of acetone allowed to react with 1 mole of the backbone amine is 2.0 mol. The “IP” in “2.0IP-TEPA” etc. indicates that two primary amino groups in the backbone amine are isopropylated, forming diisopropylamine.

Furthermore, the expression “(29)/Q30” in “TEPA(29)/Q30” etc. indicates that the polyamine content in the polyamine-containing material is 29 mass %, and the support is Q30. Q30 is a mesoporous silica (Cariact Q30: specific surface area 104 m²/g, pore volume 1.0 mL/g) manufactured by Fuji Silysia Chemical Ltd.

### <Synthesis of Polyamine>

### (1) Synthesis of BDAPZ

To 1 mole of BAPZ (1,4-Bis(3-aminopropyl) piperazine) dissolved in 800 mL of methanol, two moles of acrylonitrile was added, stirred at 25° C. for 60 minutes, and further stirred at 20° C. for 16 hours. The solvent was removed under reduced pressure, to prepare CN-BAPZ (3,3′((piperazine-1,4-diylbis(propane-3,1-diyl))bis(azanediyl))dipropanenitrile) in a transparent liquid state. The reaction scheme is shown below.

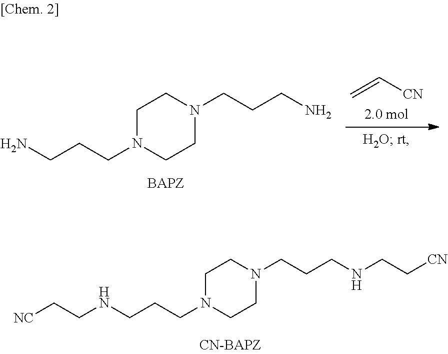

The structure of CN-BAPZ was identified by nuclear magnetic resonance spectroscopy (NMR) and liquid chromatography-mass spectrometry (LC-MS).

¹H NMR (CDCl₃, 400 MHz) δ_{H} ppm: 2.91 (4H, t), 2.67 ((4H, t), 2.51 (4H, t), 2.48-2.42 (8H, m), 2.41 (4H, t), 1.68 (4H, qn), 1.58 (2H, bs, s); ¹³C NMR (CDCl₃, 100 MHz) δ_{C} ppm: 118.8 (CN×2), 56.7 (CH₂×2), 53.2 (CH₂×4), 47.8 (CH₂×2), 45.0 (CH₂×2), 26.7 (CH₂×2), 18.5 (CH₂×2); LC-MS (ESI⁺): actual measured value m/z 307 [M+H]⁺ with respect to the theoretical molecular weight (C₁₆H₃₀N₆) 306.

In a high-pressure autoclave, 0.16 moles of CN-BAPZ and 200 mL of methanol were placed, to which 50 g of Raney cobalt catalyst and 100 mL of an aqueous ammonia solution (27 mass %) were added, followed by reaction for 12 hours in a hydrogen atmosphere at 800 kPa. The catalyst was filtered off, and the solvent was removed under reduced pressure, followed by vacuum-drying, to prepare BDAPZ (N¹,N^{1′}-(piperazine-1,4-diylbis(propane-3,1-diyl))bis(propane-1,3-diamine)) in a transparent and viscous liquid state. The reaction scheme is shown below.

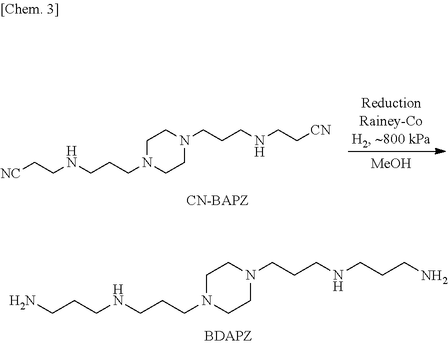 [SMILES: "MeOH" ; N#CCCNCCCN1CCN(CCCNCCC#N)CC1 ; NCCCNCCCN1CCN(CCCNCCCN)CC1]

The structure of BDAPZ was identified by NMR and LC-MS.

¹H NMR (CDCl₃, 400 MHz) δ_{H} ppm: 2.76 (4H, t), 2.68-2.61 (8H, m), 2.46-2.43 (8H, m), 2.70 (2H, m), 2.39 (4H, t), 1.71-1.66 (4H, m), 1.65-1.60 (4H, m), 1.36 (4H, bs, s); ¹³C NMR (CDCl₃, 100 MHz) δ_{C} ppm: 57.0 (CH₂×2), 53.2 (CH₂×4), 48.8 (CH₂×2), 47.8 (CH₂×2), 40.5 (CH₂×2), 33.9 (CH₂×2), 27.0 (CH₂×2); LC-MS (ESI⁺): actual measured value m/z 315 [M+H]⁺ with respect to the theoretical molecular weight (C₁₆H₃₈N₆) 314.

### (2) Synthesis of 1.0IP-BDAPZ

In a high-pressure autoclave, 0.24 mol of BDAPZ and 250 mL of ethanol were placed, to which 400 mg of PtO₂ catalyst and 0.24 mol of acetone were added, followed by reaction for 10 hours in a hydrogen atmosphere at 350 kPa. The catalyst was filtered off, and the solvent was removed under reduced pressure, followed by vacuum-drying, to prepare 1.0IP-BDAPZ (N¹-(3-(4-(3-((3-aminopropyl)amino)propyl)piperazin-1-yl)propyl)-N³-isopropylpropane-1,3-diamine) in a transparent liquid state. The reaction scheme is shown below.

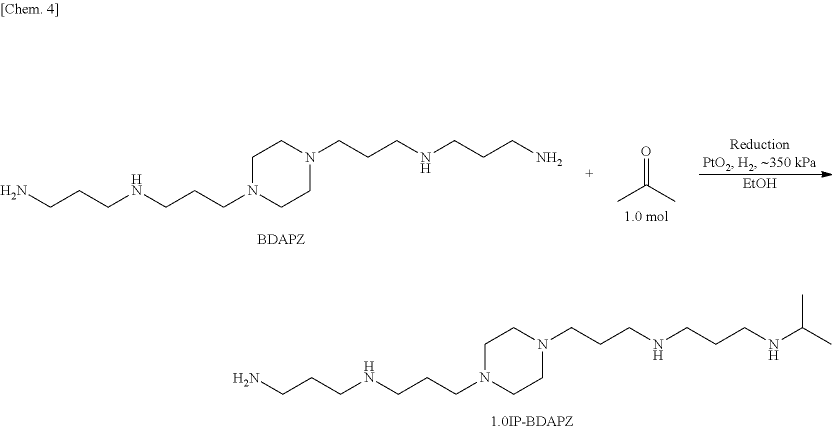

The structure of 1.0IP-BDAPZ was identified by NMR and LC-MS.

¹H NMR (CDCl₃, 400 MHz) δ_{H} ppm: 2.78 (1H, m), 2.67-2.65 (2H, m), 2.64-2.62 (10H, m), 2.49-2.43 (8H, m), 2.39 (4H, t), 1.71-1.69 (2H, m), 1.67-1.64 (4H, m), 1.63-1.60 (2H, m), 1.33 (4H, bs, s), 1.04 (6H, d); ¹³C NMR (CDCl₃, 100 MHz) δ_{C} ppm: 57.0 (CH₂×2), 53.3 (CH₂×4), 48.8 (CH₂×2), 48.7 (CH), 47.8 (CH₂×2), 48.5 (CH₂), 47.9 (CH₂), 46.1 (CH₂), 40.5 (CH₂), 33.9 (CH₂), 30.7 (CH₂×2), 27.1 (CH₂), 23.0 (CH₃×2); LC-MS (ESI⁺): actual measured value m/z 357 [M+H]⁺ with respect to the theoretical molecular weight (C₁₉H₄₄N₆) 356.

### (3) Synthesis of 2.0IP-BDAPZ

In a high-pressure autoclave, 0.24 mol of BDAPZ and 250 mL of ethanol were placed, to which 800 mg of PtO₂ catalyst and 0.48 mol of acetone were added, followed by reaction for 18 hours in a hydrogen atmosphere at 350 kPa. The catalyst was filtered off, the solvent was removed under reduced pressure, followed by vacuum-drying, to prepare 2.0IP-BDAPZ (N¹,N^{1′}-(piperazine-1,4-diylbis(propane-3,1-diyl))bis(N³-isopropylpropane-1,3-diamine)) in a transparent liquid state. The reaction scheme is shown below.

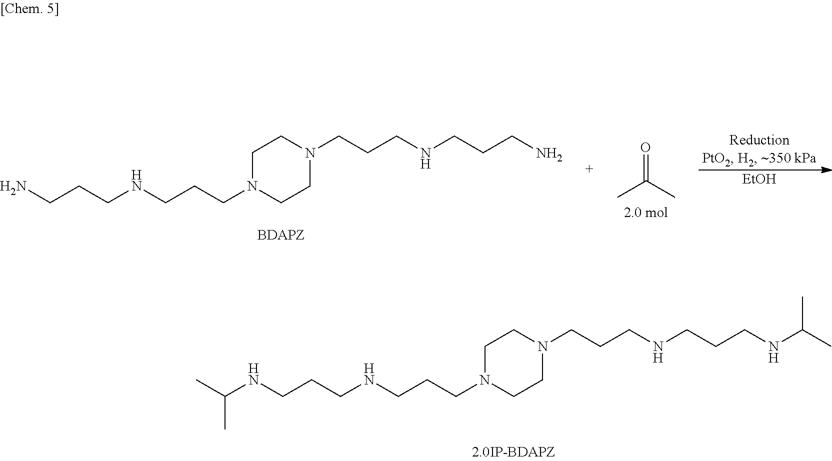

The structure of 2.0IP-BDAPZ was identified by NMR and LC-MS.

¹H NMR (CDCl₃, 400 MHz) δ_{H} ppm: 2.82-2.73 (2H, m), 2.67-2.62 (12H, m), 2.56-2.42 (8H, m), 2.38 (4H, t), 1.71-1.67 (4H, m), 1.66-1.62 (4H, m), 1.34 (4H, bs, s), 1.04 (6H, d); ¹³C NMR (CDCl₃, 100 MHz) δ_{C} ppm: 57.0 (CH₂×2), 53.3 (CH₂×4), 48.7 (CH₂×2), 48.7 (CH×2), 48.6 (CH₂×2), 46.0 (CH₂×2), 30.7 (CH₂×2), 27.1 (CH₂×2), 23.0 (CH₃×2); LC-MS (ESI⁺): actual measured value m/z 399 [M+H]⁺ with respect to the theoretical molecular weight (C₂₂H₅₀N₆) 398.

### (4) Synthesis of EPZ

From commercially available TEPA, (N²-(2-((2-(piperazine-1-yl)ethyl)amino)ethyl)ethane-1,2-diamine) was prepared using a batch distillation column. For the preparation method, Energy Technol. 2017, 5, 1186-1190 may be referred to.

### (5) Synthesis of IP-EPZ

In a high-pressure autoclave, 0.23 mol of EPZ and 250 mL of ethanol were placed, to which 400 mg of PtO₂ catalyst and 0.23 mol of acetone were added, followed by reaction for 12 hours in a hydrogen atmosphere of 350 kPa. The catalyst was filtered off, and the solvent was removed under reduced pressure, followed by vacuum-drying, to prepare IP-EPZ (N¹-isopropyl-N²-(2-((2-(piperazin-1-yl)ethyl)amino)ethyl)ethane-1,2-diamine) in a transparent liquid state. The reaction scheme is shown below.

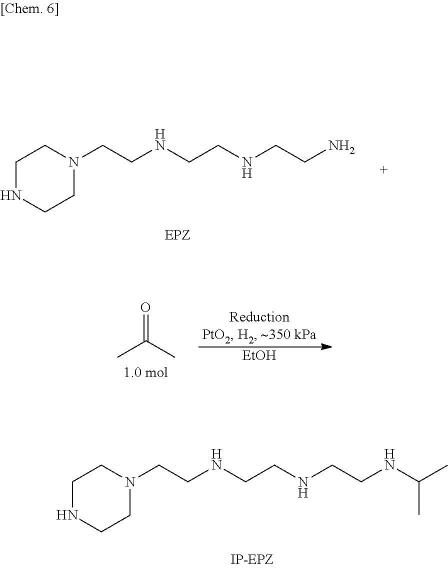

The structure of IP-EPZ was identified by NMR and LC-MS.

¹H NMR (CDCl₃, 400 MHz) δ_{H} ppm: 2.88 (4H, t), 2.78 (1H, m), 2.74-2.72 (8H, m), 2.70 (2H, br, s), 2.47 (4H, t), 2.41 (2H, br, s), 1.59 (4H, br, s), 1.06 (6H, d); ¹³C NMR (CDCl₃, 100 MHz) δ_{C} ppm: 58.2 (CH₂), 54.3 (CH₂×2), 49.4 (CH₂), 49.2 (CH₂), 49.0 (CH₂), 48.3 (CH), 46.7 (CH₂), 45.9 (CH₂), 45.8 (CH₂×2), 22.7 (CH₃×2); LC-MS (ESI⁺): actual measured value m/z 258 [M+H]⁺ with respect to the theoretical molecular weight (C₁₃H₃₁N₅) 257.

### (6) PEI

A commercially available branched polyethylene imine (PEI) having a molecular weight of 1200 (manufactured by Junsei Chemical Co., Ltd.) was prepared. An example of the structure of PEI is shown below.

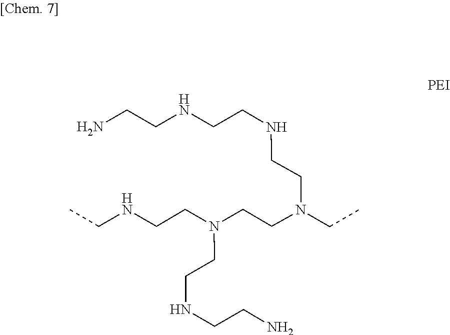

### (7) Synthesis of IP-PEI

In a flask with a capacity of 1 L, 480 mg of platinum oxide catalyst and 200 mL of commercially available anhydrous ethanol were placed, and after the atmosphere in the flask was replaced with hydrogen, hydrogen was added until a pressure of 150 kPa was reached, followed by stirring for 15 to 20 minutes at 500 rpm, to reduce the platinum oxide catalyst. Next, 200 g (0.16 mol) of polyethyleneimine (PEI-1200), 20.9 g (0.36 mol) of acetone, and 150 ml of anhydrous ethanol were placed in the flask containing the reduced catalyst. After the atmosphere in the flask was replaced with hydrogen, hydrogen was added until a pressure of about 200 kPa was reached, and the solution was stirred, until the theoretical amount (2 mol) of hydrogen was absorbed and the pressure decreased, and no absorption was observed for 10 hours thereafter. After the solution was filtered to remove the catalyst therefrom, the ethanol was removed under reduced pressure at 40° C., and the resulting colorless liquid was further dried overnight at 50° C. under vacuum, to obtain IP-PEI.

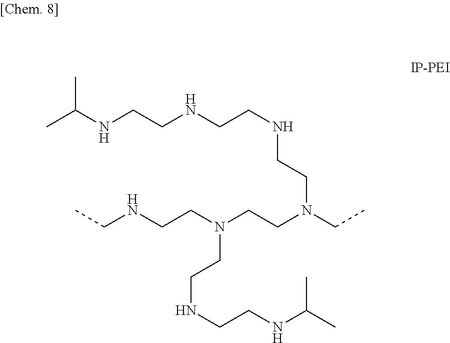

### (8) TEPA

A commercially available TEPA was used. Although commercially available TEPAs contain unavoidable impurities, such as EPZ, as mentioned above, the below shows the structural formula of TEPA as the major component.

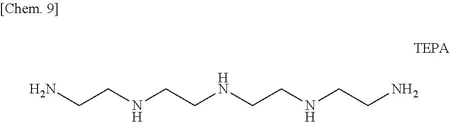 [SMILES: NCCNCCNCCNCCN]

### (9) Synthesis of IP-TEPA

In a flask with a capacity of 1 L, 2 g of platinum oxide catalyst and 100 ml of commercially available anhydrous ethanol were placed, and after the atmosphere in the flask was replaced with hydrogen, hydrogen was added until a pressure of 150 kPa was reached, followed by stirring for 15 to 20 minutes at 500 rpm, to reduce the platinum oxide catalyst. Next, 189.31 g (1.0 mol) of tetraethylenepentamine (TEPA), 127.8 g (2.2 mol) of acetone, and 150 ml of anhydrous ethanol were placed in the flask containing the reduced catalyst. After the atmosphere in the flask was replaced with hydrogen, hydrogen was added until a pressure of about 200 kPa was reached, and the solution was stirred until the theoretical amount (2 mol) of hydrogen was absorbed and the pressure decreased, and no absorption was observed for 10 hours thereafter. After the solution was filtered to remove the catalyst therefrom, the ethanol was removed under reduced pressure at 40° C., and the resulting colorless liquid was further dried overnight at 50° C. under vacuum, to obtain IP-TEPA (1,11-diisopropylamino-3,6,9-triazaundecane).

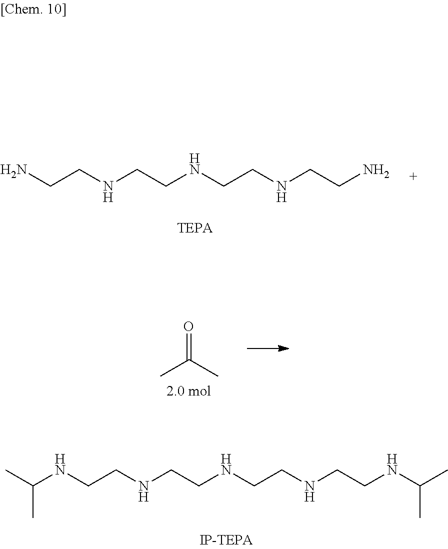

### <Preparation of Carbon Dioxide Separating Material (Polyamine-Containing Material)>

A predetermined amount of polyamine was weighed, which was dissolved in 50 mL of methanol (Wako Pure Chemical Industries, Ltd.; special grade) measured into a 200 cm³ eggplant flask. Then, 15 g of support Q30 weighed separately was placed in the eggplant flask, and stirred at room temperature for 2 hours. The mixture was heated to 40° C. in a rotary evaporator (N-1000, manufactured by EYELA) while the pressure in the system was reduced to 30 Pa, thereby removing the methanol solvent. Thus, a carbon dioxide separating material containing a predetermined amount of polyamine was obtained. The total weight of the flask and the reagents was measured in advance, and the removal of the methanol solvent was regarded as completed when a mass reduction of 20 g, which was corresponding to the mass of the methanol solvent, was confirmed. The prepared carbon dioxide separating material was stored in a desiccator, with the eggplant flask closed with a stopper, until it was used for evaluation tests.

The characteristics of the carbon dioxide separating materials are summarized in Table 1. The carbon dioxide separating materials of Examples 1 to 7 are separating materials E1 to E7, and the carbon dioxide separating materials of Comparative Examples 1 to 4 are separating materials R1 to R4.

TABLE 1

| | polyamine content | | |
|---|---|---|---|
| | (mass %) | polyamine | support |
| E1 | 25 | BDAPZ | Q30 |
| E2 | 29 | BDAPZ | Q30 |
| E3 | 35 | BDAPZ | Q30 |
| E4 | 29 | EPZ | Q30 |
| E5 | 29 | 1.0IP-BDAPZ | Q30 |
| E6 | 29 | 2.0IP-BDAPZ | Q30 |
| E7 | 29 | IP-EPZ | Q30 |
| R1 | 29 | PEI | Q30 |
| R2 | 29 | TEPA | Q30 |
| R3 | 29 | IP-PEI | Q30 |
| R4 | 29 | IP-TEPA | Q30 |

### Evaluation 1

The carbon dioxide separating material was allowed to absorb carbon dioxide by a constant volume method, and the equilibrium adsorption amounts of carbon dioxide at the respective pressures were measured. For the measurement, an automatic gas/vapor adsorption analyzer (BELSORP MAX II) manufactured by MicrotracBEL Corporation was used. About 0.1 g of a sample of the carbon dioxide separating material was weighed into a sample tube, and the sample was evacuated for 6 hours as a pretreatment. Thereafter, carbon dioxide was gradually introduced into the sample tube, to confirm the pressure at which equilibrium was reached in the range up to 100 kPa and measured the adsorption amount, at 30° C. By this method, the relationship between the partial pressure and the adsorption amount of carbon dioxide was determined. From the adsorption-desorption isotherm obtained at this time, the equilibrium adsorption amounts of carbon dioxide by the carbon dioxide separating material were read at carbon dioxide partial pressures of 0.04 kPa, 0.1 kPa, 1 kPa, 13 kPa, and 100 kPa. The results are shown in Table 2.

TABLE 2

| CO₂ separating material | | | CO₂ adsorption amount (mol/kg) | | | | |
|---|---|---|---|---|---|---|---|
| | polyamine | | (30° C.) | | | | |
| | content | | CO₂ partial pressure (kPa) | | | | |
| type | (mass %) | polyamine | 0.04 | 0.1 | 1 | 13 | 100 |
| E2 | 29 | BDAPZ | 0.86 | 1.13 | 1.31 | 1.50 | 1.74 |
| E4 | 29 | EPZ | 0.65 | 1.33 | 1.61 | 1.82 | 2.09 |
| E5 | 29 | 1.0IP-BDAPZ | 0.15 | 0.39 | 1.03 | 1.23 | 1.37 |
| E6 | 29 | 2.0IP-BDAPZ | 0.08 | 0.25 | 0.91 | 1.12 | 1.28 |
| E7 | 29 | IP-EPZ | 0.07 | 0.25 | 1.27 | 1.56 | 1.72 |
| R1 | 29 | PEI | 0.96 | 1.06 | 1.15 | 1.23 | 1.37 |
| R2 | 29 | TEPA | 0.79 | 1.13 | 1.54 | 1.60 | 1.68 |
| R3 | 29 | IP-PEI | 0.18 | 0.43 | 1.07 | 1.31 | 1.51 |
| R4 | 29 | IP-TEPA | 0.06 | 0.18 | 1.36 | 1.76 | 1.93 |

### Evaluation 2

The carbon dioxide separating material was allowed to absorb carbon dioxide by a constant volume method, and the equilibrium adsorption amounts of carbon dioxide at the respective pressures were measured. For the measurement, a high-throughput fully automatic chemical adsorption analyzer (Chemisorb HTP) manufactured by Micromeritics Corporation, which was purchased from Shimadzu Corporation, was used. About 0.1 g of a sample of the carbon dioxide separating material was weighed into a sample tube, and the sample was flowed under a helium stream at 80° C. for 6 hours as a pretreatment, and subsequently, the sample was cooled at 1° C./min to 40° C. and maintained at that temperature. Thereafter, carbon dioxide was gradually introduced into the sample tube, to confirm the pressure at which equilibrium was reached in the range up to 100 kPa, and measure the adsorption amount. By this method, the relationship between the partial pressure and the adsorption amount of carbon dioxide was determined. Then, the amount of carbon dioxide released from the sample during a pressure reduction was determined as an adsorption amount of carbon dioxide. Here, the pressure within the tube was reduced to 1.3 Pa over 20 minutes, to recover carbon dioxide. Then, the similar operation was repeated, to obtain an adsorption-desorption isotherm. From the adsorption-desorption isotherm thus obtained, the equilibrium adsorption amount of carbon dioxide by the carbon dioxide separating material at a carbon dioxide partial pressure of 13 kPa was read. The results are shown in Table 3.

TABLE 3

| CO₂ separating material | | | CO₂ adsorption | CO₂ recovery amount | recovery |
|---|---|---|---|---|---|
| | polyamine | | amount (A) | (B) (mol/kg) | rate |
| | content | | (mol/kg) | after 20 min pressure | (100B/A) |
| type | (mass %) | polyamine | 40° C./13 kPa | reduction | (%) |
| E1 | 25 | BDAPZ | 1.32 | 0.68 | 52 |
| E2 | 29 | BDAPZ | 1.49 | 0.66 | 44 |
| E3 | 35 | BDAPZ | 1.69 | 0.50 | 30 |
| E4 | 29 | EPZ | 1.98 | 0.85 | 43 |
| E5 | 29 | 1.0IP-BDAPZ | 1.35 | 1.18 | 87 |
| E6 | 29 | 2.0IP-BDAPZ | 1.20 | 1.12 | 93 |
| E7 | 29 | IP-EPZ | 1.76 | 1.72 | 98 |
| R1 | 29 | PEI | 1.65 | 0.36 | 22 |
| R2 | 29 | TEPA | 1.89 | 0.47 | 25 |
| R3 | 29 | IP-PEI | 1.20 | 1.00 | 83 |
| R4 | 29 | IP-TEPA | 1.56 | 1.53 | 98 |

### Evaluation 3

From the adsorption-desorption isotherm obtained in Evaluation 2, the equilibrium adsorption amount of carbon dioxide by the carbon dioxide separating material at a carbon dioxide partial pressure of 100 kPa was read. The results are shown in Table 4.

TABLE 4

| CO₂ separating material | | | CO₂ adsorption | CO₂ recovery amount | recovery |
|---|---|---|---|---|---|
| | polyamine | | amount (A) | (B) (mol/kg) | rate |
| | content | | (mol/kg) | after 20 min pressure | (100B/A) |
| type | (mass %) | polyamine | 40° C./100 kPa | reduction | (%) |
| E1 | 25 | BDAPZ | 1.54 | 0.89 | 58 |
| E2 | 29 | BDAPZ | 1.73 | 0.88 | 51 |
| E3 | 35 | BDAPZ | 1.96 | 0.76 | 39 |
| E4 | 29 | EPZ | 2.25 | 1.08 | 48 |
| E5 | 29 | 1.0IP-BDAPZ | 1.60 | 1.41 | 88 |
| E6 | 29 | 2.0IP-BDAPZ | 1.47 | 1.38 | 94 |
| E7 | 29 | IP-EPZ | 2.01 | 1.98 | 99 |
| R1 | 29 | PEI | 1.84 | 0.57 | 31 |
| R2 | 29 | TEPA | 2.04 | 0.62 | 30 |
| R3 | 29 | IP-PEI | 1.51 | 1.29 | 85 |
| R4 | 29 | IP-TEPA | 1.80 | 1.76 | 98 |

### Evaluation 4

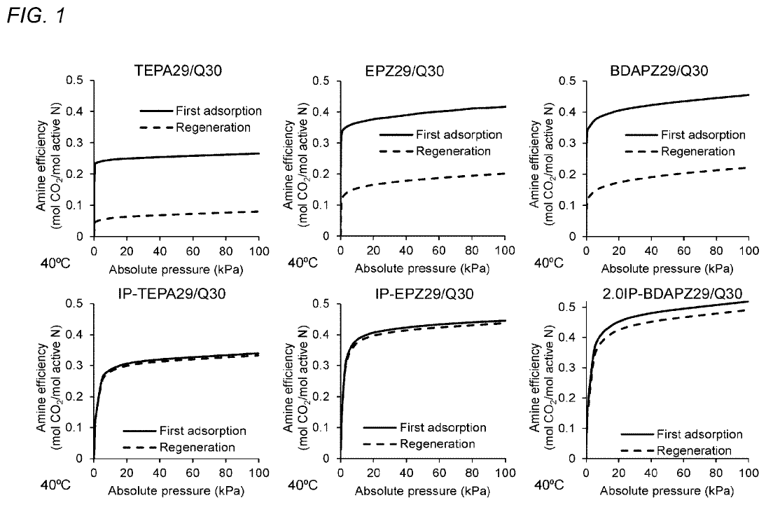

The relationship between the amine efficiency and the partial pressure of carbon dioxide was determined. The results are shown in FIG. 1. The amine efficiency is an index for evaluating the carbon dioxide absorption ability of the carbon dioxide separating material. The amine efficiency is a value expressed by a molar ratio that indicates the adsorption amount of carbon dioxide relative to the amount of nitrogen (N) contained in the carbon dioxide separating material. The amine efficiency (E) (mol−CO₂/mol−N) is calculated by the equation: E=q/n. Here, q is the amount of carbon dioxide (mol/g) adsorbed by the carbon dioxide separating material obtained in Evaluation 2, and n is the amount of mols (mol/g) of the primary or secondary amino group contained in the carbon dioxide separating material. In the absence of water vapor, theoretically, a maximum of 0.5 moles of carbon dioxide can be adsorbed per 1 mole of the primary or secondary amino group. The higher the amine efficiency is, the higher the amine utilization rate is.

### Evaluation 5

### <Oxidative Deterioration Test of Carbon Dioxide Separating Material>

An oxidative deterioration test of the carbon dioxide separating material was performed using a packed bed reaction system. First, 1.0 g of the carbon dioxide separating material was packed into a quartz tube (inner diameter 7.0 mm, outer diameter 9.5 mm, length 14 cm) placed in an oven equipped with a temperature controlling device. In order to remove gases previously adsorbed in the reaction system, the carbon dioxide separating material was heated at 100° C. for 2 hours under a nitrogen gas flow (flow rate 40 cm³/min). Next, the carbon dioxide separating material was heated at 100° C. for 42 hours under a simulated air (21% O₂, balance gas N₂) flow (flow rate 40 cm³/min), to be oxidatively deteriorated. Next, the circulation gas was switched to a nitrogen gas flow (flow rate 40 cm³/min), and the packed bed reaction system was cooled to room temperature, to obtain the deteriorated carbon dioxide separating material. The influence of water vapor on oxidative deterioration was also examined. The simulated air was passed through a temperature-controlled humidifier, to introduce water vapor into the simulated air. The relative humidity (RH) of the humidified gas flow was adjusted to 50%. The flow path connected to the humidifier was wrapped with a heating tape, and constantly heated to prevent condensation of water vapor. The applied oxidative deterioration conditions were labeled in the format of “air-temperature-oxidation time-RH.” For example, “air-100° C.-42 hours-RH50%” indicates that the carbon dioxide separating material was exposed to a simulated air at 100° C. with a relative humidity of 50% for 42 hours.

### <Carbon Dioxide Adsorption Amount Before and After Oxidative Deterioration of Carbon Dioxide Separating Material>

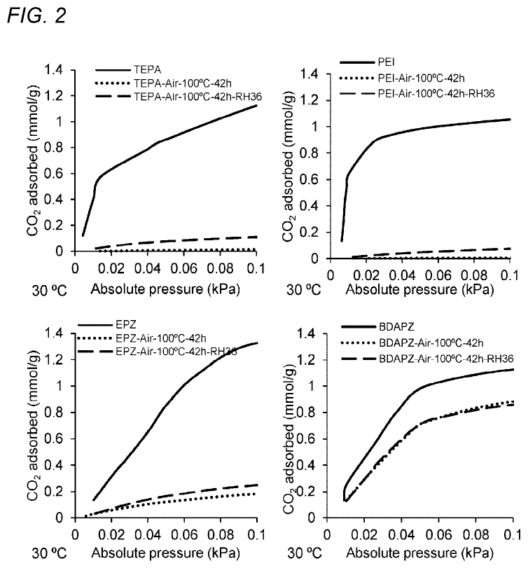

The carbon dioxide separating material before and after oxidative deterioration was allowed to absorb carbon dioxide by a constant volume method, and the equilibrium adsorption amounts of carbon dioxide at the respective pressures were measured. For the measurement, an automatic gas/vapor adsorption analyzer (BELSORP MAX II) manufactured by MicrotracBEL Corporation was used. About 0.1 g of a sample of the carbon dioxide separating material was weighed into a sample tube, and the sample was evacuated for 6 hours as a pretreatment, and maintained at 30° C. Thereafter, carbon dioxide was gradually introduced into the sample tube, to confirm the pressure at which equilibrium was reached in the range up to 0.1 kPa and measure the adsorption amount. By this method, the relationship between the partial pressure and the adsorption amount of carbon dioxide was determined. The results are shown in FIG. 2.

### Evaluation 6

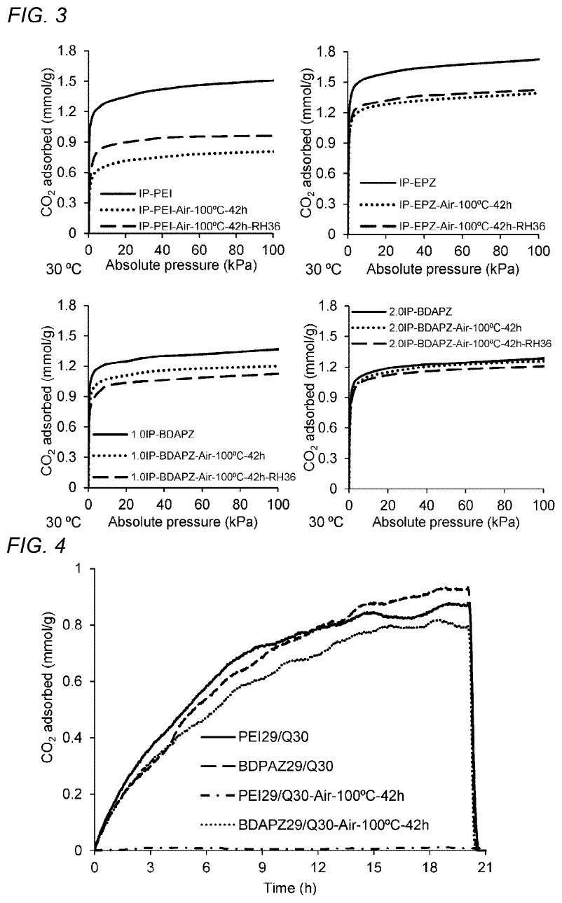

The relationship between the partial pressure and the adsorption amount of carbon dioxide was determined in the same manner as in Evaluation 5, except that the range of the pressure at which equilibrium was reached was set to be up to 100 kPa. The results are shown in FIG. 3.

### Evaluation 7

The carbon dioxide adsorption ability of the carbon dioxide separating material before and after oxidative deterioration was evaluated, using STA 449 F5 Jupiter manufactured by NETSZCH Corporation, as a thermogravimetric analyzer (TGA). In the adsorption test, about 30 to 50 mg of the carbon dioxide separating material was placed in the furnace of the TGA analyzer, and the sample was heated to 80° C. in a nitrogen gas flow, to remove the previously adsorbed carbon dioxide and moisture. The temperature was kept constant until the sample mass was stabilized. Next, the sample was cooled to 30° C., and then, a nitrogen balance gas containing 400 ppm of carbon dioxide was circulated within the furnace of the TGA analyzer for 20 hours. For the release of carbon dioxide, nitrogen at 90° C. was circulated within the furnace for 2 hours. In all the experiments, the gas flow rate was set to 50 mL/min. The mass increase was recorded, and converted to the adsorption amount of carbon dioxide, which was normalized by the dry mass of the sample. All the experiments were repeated three times in order to evaluate the reproducibility. The results are shown in FIG. 4.

### Evaluation 8

### <Study on Oxidation of Polyamines by FT-IR>

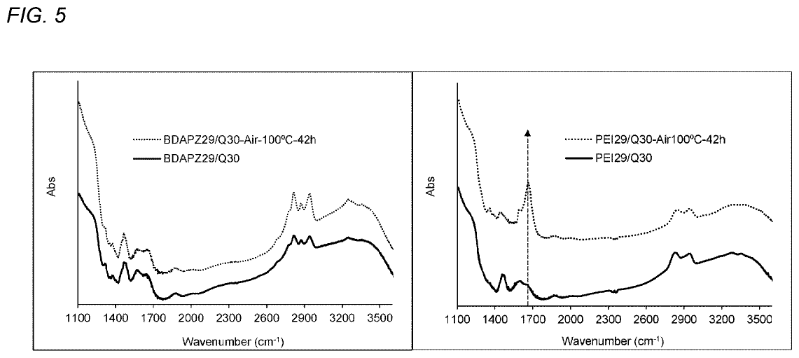

Fourier transform infrared spectra were measured at room temperature. The spectrometer used here was a Fourier transform infrared spectrophotometer (IRPrestige-21) with a resolution of 4 cm⁻¹ manufactured by Shimadzu Corporation. The IR spectra of Q30 impregnated with PEI or BDAPZ before and after oxidative deterioration are shown in FIG. 5. In the case of PEI29/Q30, a strong peak appears at 1669 cm⁻¹ after oxidative deterioration. This peak is attributed to the stretching vibration of the C═N or C═O group. This oxidation product has a correlation with an abrupt drop of the carbon dioxide adsorption ability. On the other hand, in the case of BDAPZ29/Q30, no strong peak appears at 1669 cm⁻¹ after oxidative deterioration. This indicates that BDAPZ is barely oxidized. Thus, the carbon dioxide adsorption ability of BDAPZ29/Q30 is not reduced so significantly even when exposed to a strong oxidizing atmosphere.

### Evaluation 9

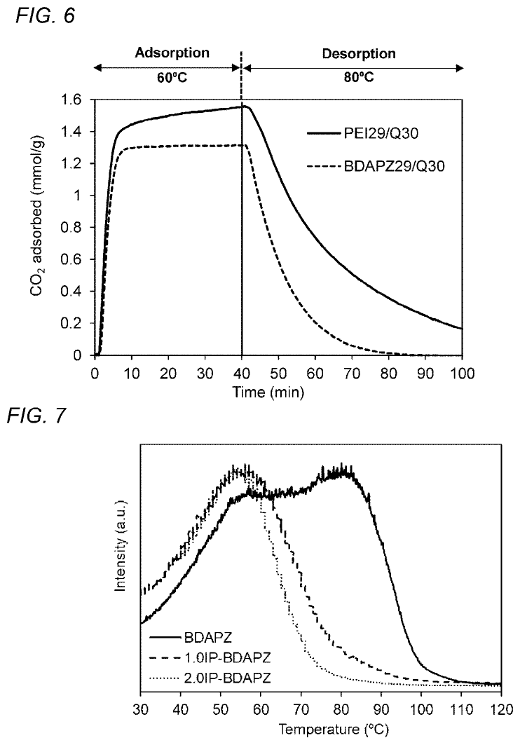

The adsorption and release of carbon dioxide by the carbon dioxide separating material was kinetically evaluated, using STA 449 F5 Jupiter manufactured by NETSZCH Corporation, as a thermogravimetric analyzer (TGA). In the adsorption test, about 30 to 50 mg of the carbon dioxide separating material was placed in the furnace of the TGA analyzer, and the sample was heated to 80° C. in a nitrogen gas flow, to remove the previously adsorbed carbon dioxide and moisture. The temperature was kept constant until the sample mass was stabilized. Next, the sample was cooled to 60° C., and then, a nitrogen balance gas containing 13% of carbon dioxide was circulated within the furnace of the TGA analyzer for 40 minutes. For the release of carbon dioxide, nitrogen at 80° C. was circulated within the furnace for 60 minutes. In all the experiments, the gas flow rate was set to 50 mL/min. The mass change was recorded, and converted to the adsorption-release amount of carbon dioxide, which was normalized by the dry mass of the sample. All the experiments were repeated three times in order to evaluate the reproducibility. The results are shown in FIG. 6.

### Evaluation 10

The carbon dioxide release ability of the carbon dioxide separating material containing a polyamine having an isopropyl group was evaluated by CO₂ temperature-programmed desorption analysis (CO₂-TPD). For the evaluation, a chemical adsorption analyzer (BELCAT-II) equipped with a gas analyzer BELMASS (manufactured by MicrotracBEL Corporation) was used. After pretreated by evacuation, the sample was saturated with carbon dioxide at 30° C., to allow carbon dioxide to be adsorbed onto the sample. Then, the sample was heated to 120° C. (temperature raising rate 2° C./min) under the circulation of helium at 30 mL/min, to perform CO₂-TPD analysis. The data was quantified using a thermal conductivity detector (TCD), and the fragment peak at m/z=44 was used to measure the released carbon dioxide. The results are shown in FIG. 7. The results confirmed that the carbon dioxide separating materials containing 1.0IP-BDAPZ and 2.0IP-BDAPZ both having at least one isopropyl group released carbon dioxide at a lower temperature than the carbon dioxide separating material containing BDAPZ having no isopropyl group.

### Evaluation 11

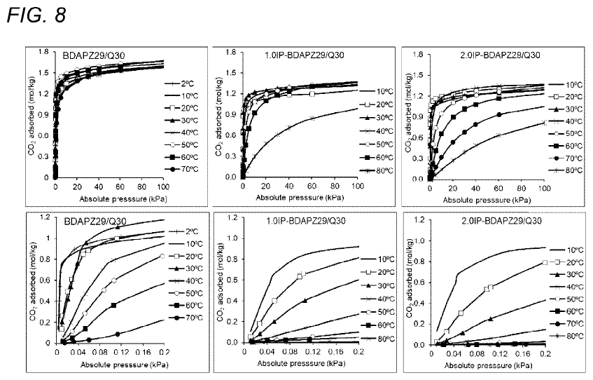

The equilibrium adsorption amounts of carbon dioxide at the respective pressures were measured using a constant volume method, thereby to evaluate the amount of carbon dioxide adsorbed onto the carbon dioxide separating material. For the evaluation, BELSORP MAX II (manufactured by MicrotracBEL Corporation) was used. About 0.1 g of a sample of the carbon dioxide separating material was weighed into a sample tube, and the sample was evacuated for 6 hours as a pretreatment. Next, the sample was maintained at an adsorption temperature (2° C. to 80° C.), and carbon dioxide was gradually introduced into the sample tube. The pressure at which equilibrium was reached was confirmed in the range up to 100 kPa, and the adsorption amount was measured. In this manner, with the adsorption temperature changed, the relationship between the partial pressure of carbon dioxide and the adsorption amount of carbon dioxide was determined. The graphs in the upper row of FIG. 8 show the data in the range up to 100 kPa, and those in the lower row show the data in the range up to 0.2 kPa. In the carbon dioxide separating materials containing 1.0IP-BDAPZ and 2.0IP-BDAPZ both having at least one isopropyl group, the lower the carbon dioxide adsorption temperature was, the more the adsorption amount tended to increase. In the carbon dioxide separating material containing BDAPZ having no isopropyl group, a similar behavior was observed under low pressure, but as the pressure increased, the difference in the adsorption amount depending on the adsorption temperature was narrowed.

### INDUSTRIAL APPLICABILITY

The carbon dioxide separating material according to the present disclosure is excellent in resistance to oxidative deterioration and enables the separation and recover of a large amount of carbon dioxide by pressure reduction in a short time, and is, therefore, efficient and practical, and suitable for reuse. Furthermore, with the carbon dioxide separating material according to the present disclosure, in the carbon dioxide absorption and release process, either a pressure swing method or a temperature swing method can be adopted for separation and recovery of carbon dioxide, and the carbon dioxide absorption and release process suitable for various usage environments can be selected. Furthermore, there is little or no deterioration in the absorption, release, and reabsorption performance even in the co-presence of water vapor, and no dehumidification process is necessary. Therefore, the construction of an energy-saving system and the miniaturization of apparatus leading to cost reduction are possible.

Although the present invention has been described in terms of the presently preferred embodiments, it is to be understood that such disclosure is not to be interpreted as limiting. Various alterations and modifications will no doubt become apparent to those skilled in the art to which the present invention pertains, after having read the above disclosure. Accordingly, it is intended that the appended claims be interpreted as covering all alterations and modifications as fall within the true spirit and scope of the invention.

1. A polyamine, comprising a ring-containing polyamine having a piperazine ring, wherein
at least one of two nitrogen atoms of the piperazine ring is bonded to a chain substituent represented by
-(A1-NR1)ₘ-X,
where m represents an integer of 2 to 50,
A1 represents an alkylene group having 2 to 6 carbon atoms,
a plurality of A1 may be identical or different from each other,
R1 is a hydrogen atom, an alkyl group having 1 to 6 carbon atoms, or an alkylamino group having 1 to 6 carbon atoms,
a plurality of R1 may be identical or different from each other, and at least one R1 is a hydrogen atom or an alkylamino group having 1 to 6 carbon atoms, and
X is a hydrogen atom, an alkyl group having 1 to 6 carbon atoms, or an alkylamino group having 1 to 6 carbon atoms.

2. The polyamine according to claim 1, where X is a hydrogen atom or an isopropyl group.

3. The polyamine according to claim 1 wherein
m represents an integer of 2 to 6,
a plurality of A1 is identical, and is an ethylene group (—CH₂CH₂—) having 2 carbon atoms or a propylene group (—CH₂CH₂CH₂—) having 3 carbon atoms, and
a plurality of R1 is a hydrogen atom.

4. The polyamine according to claim 1, including at least one selected from the group consisting of ring-containing polyamines represented by

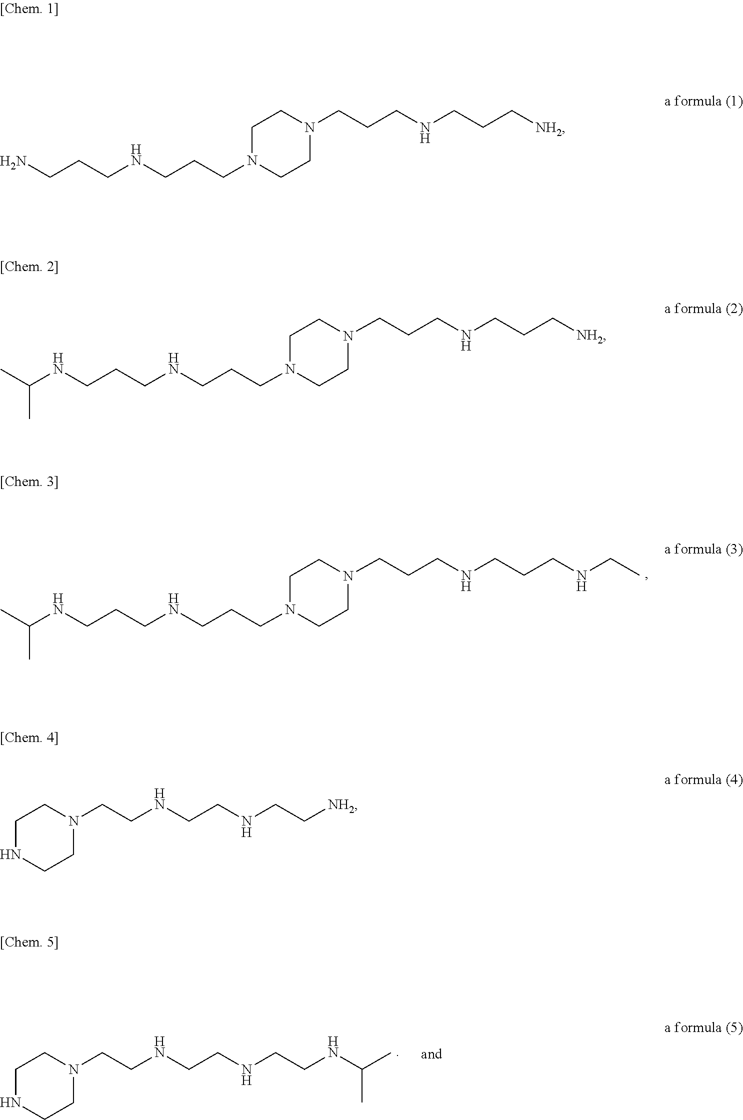 [SMILES: NCCCNCCCN1CCN(CCCNCCCN)CC1 ; CC(C)NCCCNCCCN1CCN(CCCNCCCN)CC1 ; CCNCCCNCCCN1CCN(CCCNCCCNC(C)C)CC1 ; NCCNCCNCCN1CCNCC1 ; CC(C)NCCNCCNCCN1CCNCC1]

5. The polyamine according to claim 1, wherein a ratio of a number of moles of an isopropyl group included in the ring-containing polyamine to a number of moles of the ring-containing polyamine is 0.25 to 0.75.

6. The polyamine according to claim 1, wherein a content of the ring-containing polyamine is more than 6 mass %.

7. The polyamine according to claim 1, wherein
the polyamine further includes a chain polyamine,
the chain polyamine has a structure represented by
Y-(A2-NR2)ₘ-Y,
where m represents an integer of 2 to 50,
A2 represents an alkylene group having 2 to 6 carbon atoms,
a plurality of A2 may be identical or different from each other,
R2 is a hydrogen atom, an alkyl group having 1 to 6 carbon atoms, or an alkylamino group having 1 to 6 carbon atoms,
a plurality of R2 may be identical or different from each other, and at least one R2 is a hydrogen atom or an alkylamino group having 1 to 6 carbon atoms,
Y is a hydrogen atom, an alkyl group having 1 to 6 carbon atoms, or an alkylamino group having 1 to 6 carbon atoms, and
two Y's may be identical or different from each other.

8. The polyamine according to claim 7, wherein a difference between a formula weight of the ring-containing polyamine and a formula weight of the chain polyamine is equivalent to a formula weight of an ethylene group (—CH₂CH₂—).

9. The polyamine according to claim 7, wherein a backbone amine of the chain polyamine is at least one selected from the group consisting of homopolymers of ethyleneimine, propyleneimine, 2-ethylaziridine, 2-propylaziridine, and 2-butylaziridine, and a copolymer of at least two of these.

10. The polyamine according to claim 1 wherein a backbone amine of the chain polyamine is at least one selected from the group consisting of tetraethylenepentamine, spermine, N,N,N′,N′-tetrakis(3-aminopropyl)-1,4-butanediamine, pentaethylenehexamine, hexaethyleneheptamine, and triethylenetetramine.

11. The polyamine according to claim 1, having a boiling point of 320° C. or higher at 760 mmHg.

12. A carbon dioxide separating material, comprising a polyamine-containing material including a support and the polyamine according to claim 1 carried thereon.

13. The carbon dioxide separating material according to claim 12, wherein the support is at least one selected from the group consisting of silica, polymethyl methacrylate, alumina, silica alumina, clay minerals, cordierite, magnesia, zirconia, zeolite, zeolite-related compounds, natural minerals, waste solids, activated carbon, cellulose, and carbon molecular sieves.

14. The carbon dioxide separating material according to claim 12, wherein the support has a specific surface area (BET) of 50 m²/g or more and 1000 m²/g or less, and a pore volume of 0.1 cm³/g to 2.3 cm³/g.

15. The carbon dioxide separating material according to claim 1, including the polyamine-containing material, and a binder for granulating the polyamine-containing material.

16. The carbon dioxide separating material according to claim 15, wherein the binder is at least one selected from the group consisting of silica, alumina, silica alumina, clay minerals, fluorocarbon resins, cellulose derivatives, and epoxy resins.

17. A method for separating or recovering carbon dioxide, comprising:
a first step of bringing a target gas into contact with the carbon dioxide separating material of any one of claims 12 to 16, to allow carbon dioxide to be absorbed into the carbon dioxide separating material; and
a second step of releasing carbon dioxide from the carbon dioxide separating material into which the carbon dioxide has been absorbed in the first step, wherein
the second step includes one or more of:
(A) a process of placing the carbon dioxide separating material under reduced pressure conditions, to release carbon dioxide (pressure swing method),
(B) a process of bringing at least one of water vapor and an inert gas into contact with the carbon dioxide separating material, to release carbon dioxide, and
(C) a process of heating the carbon dioxide separating material, to release carbon dioxide (temperature swing method).

18. The method for separating or recovering carbon dioxide according to claim 17, wherein the target gas is a gas having a temperature of 10° C. to 60° C., and a carbon dioxide partial pressure of 100 kPa or less.
